(19) United States
(12) Patent Application Publication
(10) Pub. No.: US 2021/0220766 A1
(43) Pub. Date: Jul. 22, 2021

(54) AIR CIRCULATING TRASH CAN HAVING ANTIBACTERIAL AND DEODORIZING FUNCTIONS

(71) Applicant: DEOCUBE CO., LTD., Seoul (KR)

(72) Inventor: Chang Whan AN, Seoul (KR)

(73) Assignee: DEOCUBE CO., LTD., Seoul (KR)

(21) Appl. No.: 16/759,324

(22) PCT Filed: Oct. 26, 2018
(86) PCT No.: PCT/KR2018/012862

§ 371 Date: Apr. 25, 2020

(30) Foreign Application Priority Data
Oct. 27, 2017 (KR) 10-2017-0141071

(51) Int. Cl. B01D 46/00; B65F 1/02; B65F 1/16; B01D 53/00; B01D 53/88; B01D 46/42; B01D 53/04; A61L 9/014; A61L 9/20

(52) U.S. Cl. CPC B01D 46/0038; A61L 2209/22; B65F 1/1607; B01D 46/0036; B01D 46/0028; B01D 46/0005; B01D 53/007; B01D 53/885; B01D 46/4227; B01D 53/04; A61L 9/014; A61L 9/205; B65F 2210/129; B65F 2210/181; B01D 2255/20707; B01D 2257/90; B01D 2259/804; B01D 2253/102; B01D 2259/4508; A61L 2209/14; B65F 1/02

(57) ABSTRACT

An air circulating trash can having antibacterial and deodorizing functions includes a trash can body having an open top and a lid coupled to the top of the trash can body to open and close the same. A filter mounting part is formed on the lid, and a functional mesh net filter having antibacterial and deodorizing functions is fixedly provided thereon so that air inside the trash can body and air outside circulate via the functional mesh net filter. During ventilation, the functional mesh net filter absorbs and decomposes the odor and bacteria inside the trash can body and the odor and bacteria outside to maximally decelerate the speed of decay of trash due to ventilation, prevent growth of various germs by antibacterial effect, decrease concentration of odor leaking to the outside by absorbing and decomposing the odor, and inhibit harmful insects such as fruit flies or cockroaches from entering.

### TECHNICAL FIELD

The present invention relates to an air circulating trash can having antibacterial and deodorizing functions, and more particularly to an air circulating trash can having antibacterial and deodorizing functions in which a filter mount is formed on a lid provided at the top of a trash can body and a functional mesh net filter having antibacterial and deodorizing functions is fixedly provided on the filter mount, so that air inside the trash can and outside air circulate through the functional mesh net filter for ventilation and, during ventilation, the functional mesh net filter adsorbs and decomposes not only odor and bacteria inside the trash can but also outside odor and bacteria.

Thereby, the air circulating trash can having antibacterial and deodorizing functions is able to maximally slow decay of trash due to ventilation, prevent growth of bacteria by an antibacterial effect, decrease the concentration of odor leaking outside by adsorbing and decomposing the odor, and inhibit the entrance of harmful insects, such as fruit flies or cockroaches, which transfer bacteria by means of the functional mesh net filter, thereby being easy to hygienically maintain.

### BACKGROUND ART

In general, various types of trash cans are provided in various places, such as, for example, roadsides, parks, and playgrounds as well as houses and offices.

In the trash cans, not only is clean trash thrown away, but also is stinky trash, such as food or beverage packing materials, leftover beverages, or food waste, also thrown away.

The stinky trash, such as food or beverage packing materials, leftover beverages, or food waste, attracts fruit flies, cockroaches, or various other insects, and easily decays, giving out a bad smell and causing growth of bacteria, mold, etc., which is not good in terms of appearance or hygiene.

In particular, the trash is not picked up from the trash cans every day, and most trash cans are neglected for a certain period of time. When trash cans are provided indoors, there are many concerns around indoor air pollution due to odor and hygienic management of trash cans is difficult.

In order to solve these problems, a trash can that is adapted to ferment food waste at a high temperature and reduce the amount of food waste to be discharged has been developed and marketed. However, this type of trash can has a high production cost and a large volume and results in high energy consumption since moisture of food waste is evaporated using thermal energy.

In another conventional trash can disclosed in Korean Utility Model Publication No. 20-1997-0054685, a container with a bottom mesh plate is centrally provided in a lid to accommodate a deodorant therein and is closed with a cover during use. Such a trash can is of a closed processing type that removes only odor leaking from the trash can by a deodorant component diffusing through the mesh plate.

In addition, there are trash cans disclosed in Korean Utility Model Publication No. 20-1995-0018561 and Korean Utility Model Registration No. 10-0271422, which also have a configuration in which a deodorant is attached inside a lid of the trash can so as to remove odor inside the closed trash can.

All of the conventional trash cans described above are adapted to use the deodorant hanging inside the lid such that the deodorant removes odor inside the trash can which is closed by the lid, but deodorization in the closed condition is not efficient.

In other words, since the occurrence of odor in the closed trash can continues, but the deodorant cannot quickly remove the odor, it is not possible to completely remove the odor that continues to occur. Furthermore, the conventional trash cans described above have no function to remove outside odor, and have a problem that the odor still remains inside the trash cans and is released whenever the lid is opened.

In particular, although the air processed by the deodorant needs to be immediately discharged to the outside in order to gradually dilute and reduce the odor inside the trash cans, the conventional configuration described above has a high possibility of the odor surviving and suffers from poor removal efficiency since the removal of odor takes place in a closed space with no vent holes.

### DISCLOSURE

### Technical Problem

Therefore, the present invention has been made in view of the above problems, and it is an object of the present invention to provide a high-quality air circulating trash can having antibacterial and deodorizing functions in which a filter mount is formed on a lid provided at the top of a trash can body and a functional mesh net filter having antibacterial and deodorizing functions is fixedly provided on the filter mount, so that air inside the trash can and outside air circulate through the functional mesh net filter for ventilation and, during ventilation, the functional mesh net filter adsorbs and decomposes not only odor and bacteria inside the trash can but also outside odor and bacteria.

Thereby, the air circulating trash can having antibacterial and deodorizing functions is able to maximally slow decay of trash due to ventilation, prevent growth of bacteria by an antibacterial effect, decrease the concentration of odor leaking outside by adsorbing and decomposing the odor, and inhibit the entrance of harmful insects, such as fruit flies or cockroaches, which transfer bacteria by means of the functional mesh net filter, thereby being easy to hygienically maintain.

### Technical Solution

To achieve the object described above, according to the present invention, provided is an air circulating trash can having antibacterial and deodorizing functions, the trash can basically including a trash can body with an open top and a lid coupled to the top of the trash can body so as to be able to open and close the trash can body.

A filter mount is formed on the lid and a functional mesh net filter having antibacterial and deodorizing functions is fixedly provided on the filter mount.

Thereby, air inside the trash can body and outside air circulate through the functional mesh net filter for ventilation and, during ventilation, the functional mesh net filter adsorbs and decomposes not only odor and bacteria inside the trash can body but also outside odor and bacteria to maximally slow decay of trash due to ventilation, to prevent growth of bacteria by an antibacterial effect, and to prevent odor from leaking outside by adsorbing and decomposing the odor.

### Advantageous Effects

In an air circulating trash can having antibacterial and deodorizing functions of the present invention, unlike conventional trash cans in which a trash can body is closed by a lid such that the flow of air is blocked, with the use of a functional mesh net filter fixedly provided on a filter mount formed on a lid using a filter fixing plate or a functional mesh net filter included in a filter assembly, the air inside a trash can body and the outside air circulate through the functional mesh net filter for ventilation, and during ventilation, the functional mesh net filter adsorbs and decomposes not only the odor and bacteria inside the trash can body, but also the outside odor and bacteria.

In this way, the air circulating trash can having antibacterial and deodorizing functions is able to decrease the concentration of odor leaking outside, maximally slow decay of trash, prevent growth of bacteria, mold, etc. by an antibacterial effect, and inhibit the entrance of harmful insects, such as fruit flies or cockroaches, which transfer bacteria by the functional mesh net filter, thereby creating a very clean environment in terms of appearance or hygiene.

In addition, when the functional mesh net filter gets dirty during use, it is possible to wash the functional mesh net filter by separating the filter fixing plate from the filter mount of the lid or by disassembling the filter assembly.

In addition, when it is suspected that the antibacterial or deodorizing function has deteriorated during use, by exposing the functional mesh net filter to sunlight in addition to washing thereof, the deodorizing and decomposing effects may be regenerated via generation of a large amount of anions by ultraviolet rays.

Accordingly, since the present invention is very convenient to use and exhibits various effects in a complex manner, it is clear that the present invention is a very innovative invention that can be safely used by a user for a long time.

### DESCRIPTION OF DRAWINGS

FIG. 1 is an exploded perspective view illustrating the entire configuration of the present invention.

FIG. 2 is an assembled perspective view of the present invention.

FIGS. 3 to 5 illustrate various modified shapes of a trash can according to the present invention.

FIG. 6 is a side cross-sectional view of the present invention.

FIG. 7 is an exploded perspective view illustrating one configuration of a filter assembly according to the present invention.

### BEST MODE

Hereinafter, one embodiment of the present invention will be described in detail with reference to the accompanying drawings. It is to be understood that the embodiment described below is given as illustrative in order to aid in understanding of the present invention, and that the present invention can be implemented in various modified ways different from the embodiment described herein. However, in the following description of the present invention, a detailed description and concrete illustration of known functions and components or known methods incorporated herein will be omitted when it is determined that a concrete description thereof may make the subject matter of the present invention rather unclear. In addition, the elements in the accompanying drawings are not necessarily drawn to scale, but may be exaggerated relative to other elements to aid in understanding of the present invention.

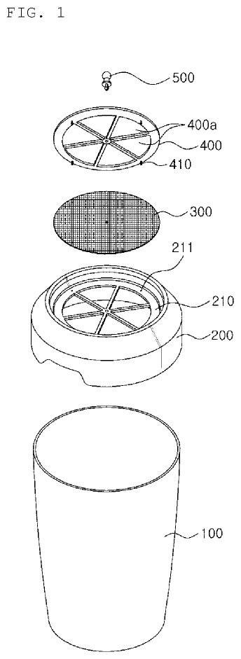

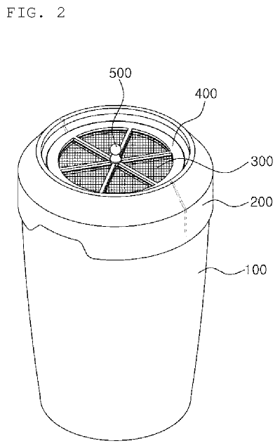

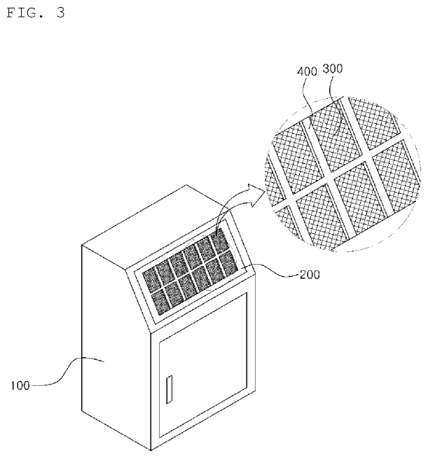

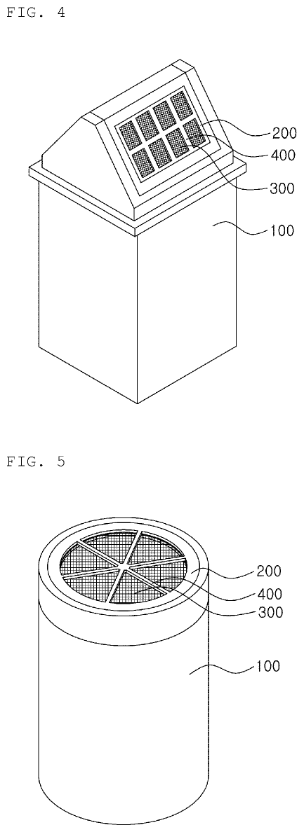

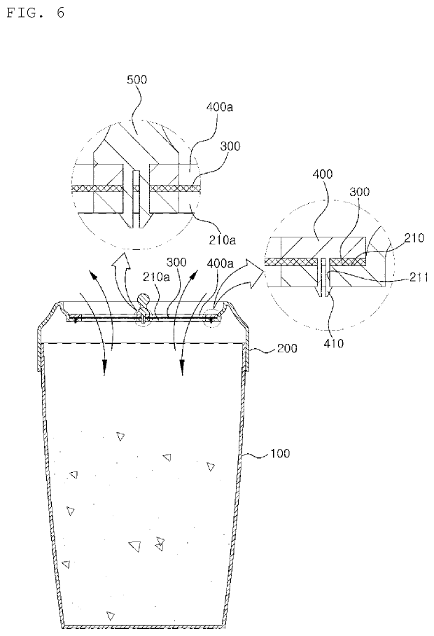

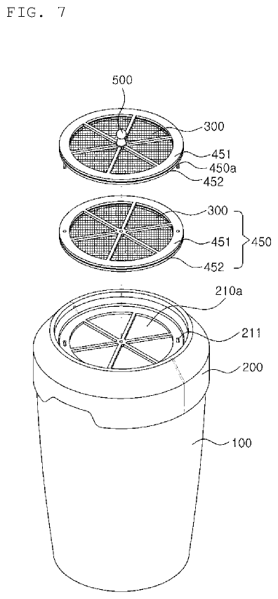

FIGS. 1 to 7 illustrate an air circulating trash can having antibacterial and deodorizing functions according to the present invention.

The trash can includes a trash can body 100 with an open top, a lid 200 coupled to the top of the trash can body 100 so as to be able to open and close the trash can body, the lid having a filter mount 210 formed thereon, and a functional mesh net filter 300 coupled to the filter mount 210 of the lid 200 by various methods.

Hereinafter, mutual systematic coupling relationships of the elements mentioned above will be described in more detail.

The trash can body 100 and the lid 200 coupled to the top of the trash can body so as to be able to open and close the trash can body have a cylindrical shape, a square shape, or a rectangular shape.

The trash can body and the lid may also be manufactured to have any of various other shapes such as, for example, an oval shape or a hexagonal shape.

Further, the lid 200 has any of various coupling structures. For example, the lid may be configured so as to be completely separated and opened away from the trash can body 100 by being lifted upward from the top of the trash can body, or may be integrally coupled to the trash can body 100 via a hinge so as to be opened away from the trash can body by being rotated about the hinge.

The filter mount 210 formed on the upper surface of the lid 200 having any of various shapes serves to support the functional mesh net filter 300 seated thereon. The filter mount 210 has at least one through-hole 210a formed therein to enable the free circulation of air through the functional mesh net filter 300.

Multiple coupling recesses 211 are formed in various shapes in the rim of the filter mount 210. The coupling recesses 211 are used for detachable coupling of a filter fixing plate 400, which will be described below.

The filter fixing plate 400 serves to aid the functional mesh net filter 300 seated on the filter mount 210 in being fixedly provided on the filter mount. The filter fixing plate has multiple through-holes 400a formed therein to enable the free circulation of air through the functional mesh net filter 300. Coupling protrusions 410 which are to be fitted into the respective coupling recesses 211 formed in the filter mount 210 protrude from the rim of the bottom surface of the filter fixing plate.

The functional mesh net filter 300 used in the present invention is manufactured to have antibacterial and deodorizing functions. For example, the functional mesh net filter is manufactured by mixing polyester yarns with an activated carbon powder, which is capable of adsorbing odor particles, and a natural mineral powder, which is capable of generating a large amount of active oxygen, such as anions or hydroxyl radicals (OH—), when stimulated by ultraviolet rays, light, wind, etc. to thereby be able to oxidatively decompose odor into water and carbon dioxide, and then performing a post treatment by any of various methods such as, for example, coating in order to prevent the leakage of contents.

Here, the natural mineral powder used in the functional mesh net filter 300 may be simply composed of any one of zeolite, bentonite, sericite, and diatomite, or a mixture thereof.

More specifically, the natural mineral powder may be composed of a slurry-type inorganic antibacterial agent prepared using zeolite, bentonite, sericite, and diatomite, and an inorganic antibacterial photocatalyst powder of synthetic rutile-type titanium oxide (TiO₂).

The synthetic rutile-type titanium oxide (TiO₂) is prepared by synthesizing titanium oxide (TiO₂) having a rutile-type crystalline structure that exhibits good heat resistance and durability so as to improve antibacterial, deodorizing, and adsorption functions thereof.

As described above, the present invention has a feature that, after at least one functional mesh net filter 300 is seated on the filter mount 210 formed on the lid 200, the filter fixing plate 400 is used to aid the functional mesh net filter in being fixedly provided on the filter mount, so that the air inside the trash can body 100 and the outside air circulate through the functional mesh net filter 300 for ventilation, and during ventilation, the functional mesh net filter 300 adsorbs and decomposes not only the odor and bacteria inside the trash can body 100 but also the outside odor and bacteria to maximally slow decay of trash due to ventilation, to prevent growth of bacteria by an antibacterial effect, and to decrease the concentration of odor leaking outside by adsorbing and decomposing the odor.

Alternatively, in the present invention, in order to ensure that the functional mesh net filter 300 is easily fixedly provided on the filter mount 210 formed on the lid 200, as particularly illustrated in FIG. 7 by way of example, the functional mesh net filter 300 is sandwiched between an upper plate 451 and a lower plate 452, each having through-holes, to construct a modular filter assembly 450. The modular filter assembly may have coupling protrusions 450a formed on the bottom surface thereof.

As such, the functional mesh net filter may be fixedly provided by fitting the coupling protrusions 450a formed on the bottom surface of the filter assembly 450 into the coupling recesses 211 formed in the rim of the upper surface of the filter mount 210 on the lid 200.

With this configuration, an operation of fixedly providing the modular filter assembly by appropriately increasing or decreasing the number of modular filter assemblies to be fixedly provided on the filter mount 210 formed on the lid 200 according to the degree of occurrence of odor inside the trash can body 100 may be very simply and rapidly performed. Furthermore, when the functional mesh net filter 300 gets dirty after use thereof for a long period of time, an operation of disassembling and washing the functional mesh net filter for each filter assembly 450 may be very easily performed.

In the present invention, a handle 500 having any of various shapes may further be integrally formed on or detachably coupled to the upper surface of the filter fixing plate 400 or the filter assembly 450 in order to enable easy separation of the functional mesh net filter 300 from the lid 200.

### MODE FOR INVENTION

Hereinafter, the use example of the air circulating trash can having antibacterial and deodorizing functions configured as described above according to the present invention will be described.

First, after various trash is thrown into the trash can body 100 with the lid 200 being opened, the lid 200 is closed.

In the trash can of the present invention, unlike conventional trash cans in which the trash can body 100 is closed by the lid 200 such that the flow of air is blocked, with the use of the functional mesh net filter 300 fixedly provided on the filter mount 210 formed on the lid 200 using the filter fixing plate 400 or the functional mesh net filter 300 included in the filter assembly 450, the air inside the trash can body 100 and the outside air circulate through the functional mesh net filter for ventilation.

During ventilation, the functional mesh net filter 300 adsorbs and decomposes not only the odor and bacteria inside the trash can body 100, but also the outside odor and bacteria.

Accordingly, the air circulating trash can having antibacterial and deodorizing functions is able to decrease the concentration of odor leaking outside, maximally slow decay of trash, prevent growth of bacteria, mould, etc. by an antibacterial effect, and inhibit the entrance of harmful insects such as fruit flies or cockroaches by the functional mesh net filter, thereby creating a very clean environment in terms of appearance or hygiene.

The following <Table 1> shows the results of a deodorization test of the functional mesh net filter 300 used in the present invention with respect to ammonia (NH₃) gas, which is an odor causing material.

TABLE 1

| | | Test Result | | |
|---|---|---|---|---|
| | | Blank | Sample | |
| | | Concentration | Concentration | Deodorization |
| Test Item | | (μmol/mol) | (μmol/mol) | Rate (%) |
| Deodorization | 0 min | 50 | 50 | 0.0 |
| Rate (%): | 30 min | 49 | N.D. | 100.0 |
| Gas Detection | 60 min | 49 | N.D. | 100.0 |
| Tube Method | 90 min | 49 | N.D. | 100.0 |
| | 120 min | 48 | N.D. | 100.0 |

Annotation) 1. Test Condition
Sample Size: 20 cm×20 cm
Test Gas: Ammonia (NH₃)
Initial Concentration of Injected Test Gas: 50 μmol/mol
Can Volume: 5 L

2. Test Environment: Temperature 23° C.±5° C., Humidity 5%±10%

3. Note: Deodorization Rate (%)=[(Blank Gas Concentration-Sample Gas Concentration)/Blank Gas Concentration]×100

4. A separate test was performed under the absence of the sample based on the above test condition and test environment.

The concentration at this time is called “Blank Concentration”.

Referring to the following <Table 1>, it could be found that the deodorization rate gradually increases over time.

In addition to ammonia (NH₃) which is an odor causing material, the test was also performed on other odor causing materials.

As a result of performing the test on hydrogen sulfide (H₂S), acetaldehyde (CH₃CHO), methyl mercaptan (R—SH), methyl ethyl ketone (MEX), formaldehyde (HCHO), acetic acid (CH₃COOH), amine (R—NH₂), phenol (R—OH), and methane gas (CH₄), it could also be found that the deodorization rate gradually increases over time.

For reference, the deodorization reaction formulae of the odor causing materials mentioned above are shown in the following <Table 2>.

TABLE 2

| Odor Causing Material | Deodorization Reaction Formula |
|---|---|
| Ammonia (NH₃) | 4NH₃ + 3O₂ → 2N₂ + 6H₂O2NH₃ + |
| | 6OH → N₂ + 6H₂O |
| Hydrogen sulfide (H₂S) | 2H₂S + O₂ → 2H₂O + 2SH₂S + |
| | 2OH → 2H₂O + S |
| Acetaldehyde (CH₃CHO) | CH₃CHO + 6OH + O₂ → 2CO₂ + |
| | 5H₂O |
| Methyl mercaptan (R—SH) | CH₃SH + 2O₂ → CO₂ + 2H₂O + S |
| Methyl ethyl ketone (MEX) | CH₃COC₂H₅ + 1 1/2O₂ → 4CO₂ + |
| | 4H₂O |
| Formaldehyde (HCHO) | HCHO + O₂ → CO₂ + H₂OHCHO + |
| | 4OH → CO₂ + 3H₂O |
| Acetic acid (CH₃COOH) | CH₃COOH + 4OH + O₂ → 2CO₂ + |
| | 4H₂O |
| Amine (R—NH2) | 2CH₃NH2 + 9/2 O₂ → 2CO₂ + N₂ + |
| | 5H₂O |
| Phenol (R—OH) | C6H₅OH + 7O₂ → 6CO₂ + 3H₂O |
| Methane (CH₄) | CH₄ + 4OH + O₂ → CO₂ + 4H₂O |

The following <Table 3> details the results of an antibacterial test of the functional mesh network filter 300 used in the present invention with respect to Staphylococcus aureus and Klebsiella pneumoniae which are harmful bacteria.

TABLE 3

| Test Item | Antibacterial Rate | Test Result |
|---|---|---|
| Antibacterial | Test Bacteria [symbol] | 99.9 |
| Degree(%): KS | Test Bacteria [symbol] | 99.9 |
| K0693:2016 Ver. | | |

Annotation) 1. Test Bacterial Species:

Test Bacteria \textcircled{1}: Staphylococcus aureus ATCC 6538

Test bacteria \textcircled{2}: Klebsiella pneumoniae ATCC 4352

2. Concentration of Inoculation Bacterial Solution:
Test Bacteria \textcircled{1}—1.0×10⁵ CFU/ml
Test Bacteria \textcircled{2}—0.8×10⁵ CFU/ml

3. Comparison Sample: Standard Cotton Cloth

4. Nonionic Surfactant: Tween 80 is added by 0.05% of the inoculation bacterial solution

Referring to the above <Table 3>, it could be found that more than 99.99% of Staphylococcus aureus and Klebsiella pneumonia is killed.

Staphylococcus aureus causes food poisoning with abdominal pain, sickness, diarrhea, etc., and Klebsiella pneumoniae is usually found in the intestinal tract, the mouth, etc., and causes acute pneumonia. It is known that 10-20% of bronchial pneumonia is caused by Klebsiella pneumoniae.

Therefore, it can be conceived that, with the use of the air circulating trash can having antibacterial and deodorizing functions of the present invention, it is possible to prevent odor particles from leaking outside by continuously adsorbing and decomposing the odor particles, to maximally slow decay of trash, to prevent growth of bacteria, mould, etc. by an antibacterial effect, and to inhibit the entrance of harmful insects such as fruit flies or cockroaches by the functional mesh net filter, which may result in the creation of a very clean environment in terms of appearance or hygiene.

Meanwhile, when the functional mesh net filter 300 gets dirty during use of the trash can of the present invention, it is possible to wash the functional mesh net filter 300 by separating the filter fixing plate 400 from the filter mount 210 of the lid 200 or by disassembling the filter assembly 450.

In addition, when it is suspected that the antibacterial or deodorizing function deteriorates during use, by exposing the functional mesh net filter 300 to sunlight in addition to washing thereof, the deodorizing and decomposing effects may be regenerated via generation of a large amount of anions by ultraviolet rays.

Accordingly, the air circulating trash can having antibacterial and deodorizing functions of the present invention may be used safely and conveniently for a long time.

Although only one exemplary embodiment of the present invention has been described with reference to the accompanying drawings, the present invention is not limited by such an exemplary embodiment, and various modifications and substitutions related to the above description are possible by those skilled in the art based on the technical ideas of the present invention within the range equivalent to the claims below.

### DESCRIPTION OF REFERENCE NUMERALS

100: trash can body 200: lid
210: filter mount 210a, 400a: through-hole
211: coupling recess 300: functional mesh net filter
400: filter fixing plate 410, 450a: coupling protrusion
450: filter assembly 451: upper plate
452: lower plate 500: handle

1. An air circulating trash can having antibacterial and deodorizing functions, the trash can comprising:
a trash can body with an open top; and
a lid coupled to the top of the trash can body so as to be able to open and close the trash can body,
wherein:
a filter mount is formed on the lid; and
a functional mesh net filter having antibacterial and deodorizing functions is fixedly provided on the filter mount, so that air inside the trash can body and outside air circulate through the functional mesh net filter for ventilation.

2. The trash can according to claim 1, wherein:
the filter mount formed on the lid has a coupling recess formed in a rim of an upper surface thereof, and
a filter fixing plate to be coupled to the filter mount has a coupling protrusion protruding from a rim of a bottom surface thereof so as to be fitted into the coupling recess, so that, after the functional mesh net filter is seated on the upper surface of the filter mount, the coupling protrusion on the bottom surface of the filter fixing plate is fitted into the coupling recess in the filter mount to allow the functional mesh net filter to be fixedly provided on the filter mount.

3. The trash can according to claim 1, wherein:
the functional mesh net filter is sandwiched between an upper plate and a lower plate, each having a through-hole formed therein, to construct a filter assembly, and
the filter assembly has a coupling protrusion formed on a rim of a bottom surface thereof, and the filter mount formed on the lid has a coupling recess formed in a rim of an upper surface thereof, so that the coupling protrusion on the bottom surface of the filter assembly is fitted into the coupling recess in the filter mount to allow the functional mesh net filter to be fixedly provided on the filter mount.

4. The trash can according to claim 3, wherein:
the filter assembly is able to be fixedly provided by appropriately increasing or decreasing a number of filter assemblies to be fixedly provided on the filter mount formed on the lid according to a degree of occurrence of odor inside the trash can body.

5. The trash can according to claim 2, further comprising:
a handle integrally formed on or detachably coupled to an upper surface of the filter fixing plate or the filter assembly to enable easy separation of the functional mesh net filter from the lid.

6. The trash can according to claim 1, wherein:
the functional mesh net filter is manufactured to have antibacterial and deodorizing functions by mixing polyester yarns with an activated carbon powder, which is capable of adsorbing odor particles, and a natural mineral powder, which is capable of generating a large amount of active oxygen such as anions or hydroxyl radicals (OH—) when stimulated by ultraviolet rays, light, wind, etc. to thereby be able to oxidatively decompose odor into water and carbon dioxide, and then performing a post treatment in order to prevent leakage of contents.

7. The trash can according to claim 6, wherein the natural mineral powder used in the functional mesh net filter is any one of zeolite, bentonite, sericite, and diatomite, or a mixture thereof.

8. The trash can according to claim 6, wherein:
the natural mineral powder used in the functional mesh net filter includes a slurry-type inorganic antibacterial agent prepared using zeolite, bentonite, sericite, and diatomite, and an inorganic antibacterial photocatalyst powder of synthetic rutile-type titanium oxide (TiO₂) prepared by synthesizing titanium oxide (TiO₂) having a rutile-type crystalline structure that exhibits good heat resistance and durability so as to improve antibacterial, deodorizing, and adsorption functions thereof.

9. The trash can according to claim 3, further comprising:
a handle integrally formed on or detachably coupled to an upper surface of the filter fixing plate or the filter assembly to enable easy separation of the functional mesh net filter from the lid.

10. The trash can according to claim 4, further comprising:
a handle integrally formed on or detachably coupled to an upper surface of the filter fixing plate or the filter assembly to enable easy separation of the functional mesh net filter from the lid.

11. The trash can according to claim 1, wherein:
during ventilation, the functional mesh net filter adsorbs and decomposes odor and bacteria inside the trash can body and adsorbs and decomposes outside odor and bacteria, thereby:
slowing decay of trash due to ventilation,
preventing growth of bacteria by antibacterial effect, and
decreasing concentration of odor leaking outside by adsorbing and decomposing the odor.

12. The trash can according to claim 11, wherein the functional mesh net filter inhibiting entrance of insects.
